(19) United States
(12) Patent Application Publication
(10) Pub. No.: US 2025/0011270 A1
(43) Pub. Date: Jan. 9, 2025

(54) CATALYTIC METHOD FOR PRODUCTION OF ACRYLIC ACID

(71) Applicant: CARGILL, INCORPORATED, Wayzata, MN (US)

(72) Inventors: Amandine CABIAC, Givors (FR); Vincent COUPARD, Lyon (FR); Souad RAFIK-CLEMENT, Vourles (FR); Justine TIRAPU, Brix (FR)

(73) Assignee: CARGILL, INCORPORATED, Wayzata, MN (US)

(21) Appl. No.: 18/712,351

(22) PCT Filed: Nov. 21, 2022
(86) PCT No.: PCT/US2022/080216

§ 371 Date: May 22, 2024

(30) Foreign Application Priority Data
Nov. 22, 2021 (FR) FR2112295

(51) Int. Cl. C07C 51/377

(52) U.S. Cl. CPC C07C 51/377; C07C 2527/18

(57) ABSTRACT

This disclosure relates to a novel material based on potassium phosphate salt or cesium phosphate salt and silica, comprising at least one source of silica formed into shape with at least one powder of a potassium phosphate or cesium phosphate salt and the use of this material for the preparation of acrylic acid. The disclosure also relates to a process for preparing said material, comprising at least one step of mixing at least one powder of at least one source of silica with at least one powder of at least one potassium phosphate or cesium phosphate salt and at least one solvent, a step of forming into shape preferably by extrusion of the mixture obtained on conclusion of the mixing step and a step of preparing calcined extrudates and optionally a final hydrothermal treatment step.

### CROSS-REFERENCE TO RELATED APPLICATIONS

This application claims the benefit of French Application No. FR2112295, filed Nov. 22, 2021, which is incorporated by reference herein in its entirety.

### TECHNICAL FIELD

The invention relates to the use of a novel material based on potassium phosphate salt or cesium phosphate salt and silica, comprising at least one source of silica formed into shape with at least one powder of a potassium phosphate or cesium phosphate salt. The novel material is useful as a catalyst for the conversion of lactic acid to acrylic acid. The invention also relates to a process for preparing said material, comprising at least one step of mixing at least one powder of at least one source of silica with at least one powder of at least one potassium phosphate or cesium phosphate salt and at least one solvent, a step of forming into shape preferably by extrusion of the mixture obtained on conclusion of the mixing step and a step of preparing calcined extrudates and optionally a final hydrothermal treatment step.

### BACKGROUND

It is known that silica, which is an advantageous compound for use as a catalytic support, cannot be extruded like other materials in conventional extrusion equipment to give products that are sufficiently resistant to be used in processes. This is because, from its manufacture to its use, the catalyst is confronted with numerous steps that can have an impact on its physical integrity. It must notably be resistant to crushing, to attrition and to pressure variations linked to the operating conditions of the catalytic reactor in which the process is performed. Thus, there is a continual need for catalysts which have improved mechanical and physical properties.

Patent application WO 2003/026795 relates to a process for producing a silica-supported catalyst, which consists in impregnating a silica constituent with a catalytic metal by means of an aqueous alkali bath, before drying in order to improve the mechanical strength thereof. More particularly, said process consists in forming and in washing a silica constituent, of silica gel or co-gel type, for example a silica-zirconia co-gel. Next, the washed silica constituent is brought into contact with the alkali bath in order for the impregnation with the catalytic metal, of cesium type, to take place, in order to form an activated silica constituent. The activated silica constituent is then dried in order to form a catalyst. The catalysts obtained have good mechanical strength. The present invention stands out through the route of production (by kneading-extrusion vs impregnation of silica beads) and the formulation of the solids obtained.

The WO 17/040383 (U.S. Pat. No. 9,849,447) patent family claims several generations of catalysts, all composed of a mixture of alkali metal phosphates, including some of formula MxPOy (M=K or Cs), and of a non-porous siliceous binder. The material described is prepared by mechanical mixing, by means of a planetary mill, of a dense amorphous molten silica (fused silica) material, which has no surface or porosity properties, and of potassium phosphate precursors. The catalyst calcined in air at 450° C. is composed of a KPO3/(KPO3+SiO2) mixture with a mass ratio of 13 to 26 wt % of KPO3. According to Example 8, the final catalyst is in the form of a powder having a variable screened particle size of between 106 and 212 μm.

The U.S. Pat. No. 8,884,050 patent family relates to the vapor phase dehydration of lactic acid to acrylic acid.

Another object of the present invention is to provide a process for preparing said material according to the invention, said material obtained having good mechanical strength and in particular a high SPC suitable for its use in the presence of a solvent and thus in an industrial process over long periods, notably by means of the use of a hydrothermal treatment step in a preferred embodiment of the invention.

Another object of the present invention is to provide a material formed into shape which can be used as a catalytic support or as a catalyst in catalytic processes to convert lactic acid to acrylic acid.

### SUMMARY

More precisely, the present invention relates to a process for the preparation of acrylic acid comprising the steps of: i) preparing a lactic acid containing feedstock, ii) heating the feedstock to obtain a lactic acid containing vapor phase, iii) contacting the lactic acid containing vapor phase with a catalyst to create a reacted vapor phase, and iv) isolating acrylic acid from the reacted vapor phase wherein the catalyst is prepared by a process, comprising at least the following steps:
a) a step of premixing at least one colloidal silica sol with at least one powder of at least one potassium phosphate salt and/or cesium phosphate salt, so as to obtain a suspension,
b) a step of adding a powder of at least one precipitated silica and at least one solvent to said suspension obtained on conclusion of step a), so as to obtain a paste,
c) a step of forming into shape the paste obtained on conclusion of step b), and
d) a step of maturing the material obtained on conclusion of step c).

One advantage of the present invention is that of providing a process for preparing a material comprising at least two sources of silica formed into shape with at least one powder of at least one potassium phosphate salt or cesium phosphate salt having an improved mechanical strength compared with the prior art materials by virtue of the implementation of a step a) of premixing a specific silica precursor, a colloidal silica sol, with a powder of potassium phosphate salt and/or cesium phosphate salt, so as to obtain a suspension, followed by a step of adding a powder of another silica precursor, a precipitated silica, then forming into shape the mixture, then maturing the extrudates obtained, notably by virtue of the implementation of a hydrothermal treatment step in one preferred embodiment of the invention.

In one particular embodiment, the use, in the premixing step a), of a source of precipitated silica or silica gel having a reduced size preferably less than 10 μm, preferably less than 5 μm, more preferably less than 1 μm, combined with the use of a powder of at least one potassium phosphate salt and/or cesium phosphate salt milled and screened to a particle size of less than 100 μm, makes it possible to obtain a material comprising at least two sources of silica formed into shape with at least one powder of at least one potassium phosphate salt or cesium phosphate salt having a particularly improved mechanical strength compared to the prior art materials.

Another object is a process for the preparation of acrylic acid comprising the steps of: i) preparing a lactic acid containing feedstock, heating the feedstock to obtain a lactic acid containing vapor phase, contacting the lactic acid containing vapor phase with a catalyst to create a reacted vapor phase and isolating acrylic acid from the reacted vapor phase where in the catalyst is prepared by the methods disclosed herein.

### DETAILED DESCRIPTION

For the purposes of the present invention, the various embodiments presented may be used alone or in combination with each other, without any limit to the combinations.

For the purposes of the present invention, the various ranges of parameters for a given step, such as the pressure ranges and the temperature ranges, may be used alone or in combination. For example, for the purposes of the present invention, a preferred range of pressure values can be combined with a more preferred range of temperature values.

Throughout the text hereinbelow, the term “side crush strength” means the mechanical strength of the material according to the invention, determined by the single pellet crush (SPC) test. This is a standardized test (standard ASTM D4179-01) which consists in subjecting a material in the form of a millimetre-sized object, such as a bead, a pellet or an extruded object, to a compressive force generating break. This test is thus a measurement of the tensile strength of the material. The analysis is repeated on a certain number of solids taken individually and typically on a number of solids of between 10 and 200. The mean of the breaking side forces measured constitutes the mean SPC which is expressed, in the case of granules, in unit force (N), and in the case of extruded objects, in unit force per unit length (daN/mm or decaNewtons per millimetre of extruded object length).

Throughout the text hereinbelow, the term “specific surface area” means the BET specific surface area (SBET) determined by nitrogen adsorption in accordance with the standard ASTM D 3663-78 established from the Brunauer-Emmett-Teller method described in the journal The Journal of the American Chemical Society, 60, 309 (1938).

The term “macropores” means pores whose aperture is greater than 50 nm.

The term “mesopores” means pores whose aperture is between 2 nm and 50 nm, limits inclusive.

The term “total pore volume” (TPV) of the material according to the invention means the volume measured by mercury intrusion porosimetry according to the standard ASTM D4284-83 at a maximum pressure of 4000 bar (400 MPa), using a surface tension of 484 dynes/cm and a contact angle of 140°. The wetting angle was taken equal to 140° following the recommendations of the publication “Techniques de l'ingénieur, traité analyse et caractérisation” [Techniques of the Engineer, Analysis and Characterization Treatise], pages 1050-1055, written by Jean Charpin and Bernard Rasneur.

In order to obtain better accuracy, the value of the total pore volume corresponds to the value of the total pore volume measured by mercury intrusion porosimetry measured on the sample minus the value of the total pore volume measured by mercury intrusion porosimetry measured on the same sample for a pressure corresponding to 30 psi (about 0.2 MPa).

The volume of the macropores and of the mesopores is measured by mercury intrusion porosimetry according to the standard ASTM D4284-83 at a maximum pressure of 4000 bar (400 MPa), using a surface tension of 484 dynes/cm and a contact angle of 140°. The value from which the mercury fills all the intergranular voids is set at 0.2 MPa and it is considered that, above this, the mercury penetrates into the pores of the sample.

The macropore volume of the material according to the invention is defined as being the cumulative volume of mercury introduced at a pressure of between 0.2 MPa and 30 MPa, corresponding to the volume contained in the pores with an apparent diameter of greater than 50 nm.

The mesopore volume of the material according to the invention is defined as being the cumulative volume of mercury introduced at a pressure of between 30 MPa and 400 MPa, corresponding to the volume contained in the pores with an apparent diameter of between 2 and 50 nm.

The median diameter of the macropores (Dmacro in nm) is also defined as being a diameter such that all the pores of size less than this diameter constitute 50% of the mesopore volume, measured by mercury porosimetry.

In the text hereinbelow, transmission electron microscopy (TEM) is the method used to characterize the materials obtained according to the invention. This technique makes it possible to obtain information on the chemical composition and the morphology, and to measure the size of the grains or of the crystals constituting the material. For that, use is made of an electron microscope (of the JEOL JEM F200 or JEOL JEM 2100F type) equipped with an energy dispersive spectrometer (EDS). The EDS detector must allow the light elements to be detected. The combination of these two tools, TEM and EDS, makes it possible to combine the imaging and local chemical analysis with good spatial resolution.

In the text hereinbelow; the size of the grains or particle size of the constituents of the materials obtained according to the invention is measured by the laser scattering particle size analysis technique. This indirect measurement technique makes it possible to determine the size distribution of particles (scale of 1 micron to 1 millimetre). This analysis method uses the principle of light scattering (Mie theory) and/or of light diffraction (Fraunhofer theory and Mie theory). The particles illuminated by the laser light deviate the light from its main axis. The amount of light deviated and the size of the angle of deviation make it possible to accurately measure the particle size. The powder is conveyed either by a solvent (water, isopropanol) or by air before passing in front of the laser beam: two approaches are thus distinguished: wet particle size analysis and dry particle size analysis.

Wet particle size analysis makes it possible to characterize dispersions (elementary particle size analysis after dispersion) or solids in suspension (“aggregated” particle size analysis). The particles measured are in the range 0.02 to 2000 microns.

Dry particle size analysis makes it possible to characterize powders of which the initial aggregation is not destroyed. The measurement range extends from 0.2 to 2000 microns. In the present invention, dry particle size analysis is used to measure the size of the grains of the constituents of the material of the invention.

In accordance with the invention, the present invention relates to a process for preparing a material, comprising at least the following steps:
a) a step of premixing at least one colloidal silica sol with at least one powder of at least one potassium phosphate salt and/or cesium phosphate salt, so as to obtain a suspension,
b) a step of adding a powder of at least one precipitated silica and at least one solvent to said suspension obtained on conclusion of step a), so as to obtain a paste,
c) a step of forming into shape the paste obtained on conclusion of step b),
d) a step of maturing the material obtained on conclusion of step c).

### Step a):

In accordance with the invention, said step a) consists in premixing at least one colloidal silica sol with at least one powder of at least one potassium phosphate salt and/or cesium phosphate salt, so as to obtain a suspension.

A solvent, preferably water, can advantageously be added in the premixing step a).

Preferably, the colloidal silicas or silica sols are chosen, without being restricted, from the following commercial sources and are in liquid form: Ludox (W. R. Grace Davison®), Nyacol (Nyacol Nano Technologies®, Inc. or PQ Corp®), Nalco (Nalco Chemical Company®), Ultra-Sol (RESI Inc®), NexSil (NNTI®), taken individually or as a mixture.

Said source(s) of colloidal silica sol used in the process according to the present invention are advantageously amorphous synthetic silicas.

Said potassium phosphate salt(s) and/or cesium phosphate salt(s) used in step a) is (are) advantageously chosen from potassium phosphate salts or cesium phosphate salts in amorphous or crystalline oxide form, taken alone or as a mixture.

Said potassium phosphate salt(s) are advantageously chosen from the following list: KH2PO4, KH2P2O12, K6P6O7, K3H2P3O10, K4H2P4O13, K3P3O9, K4P4O12, K6P6O18, K8P8O24, K10P10O30, potassium (tripotassium) phosphate (PO43−, 3K+), alone or as a mixture. Preferably, the preferred potassium phosphate salt is KH2PO4.

Said cesium phosphate salt(s) are advantageously chosen from the following list: CsH2PO4, Cs2H2P3O10, Cs4H2P4O13, Cs3P3O9, Cs4P4O12, Cs6P6O18, Cs8P8O24, (CsPO3), alone or as a mixture. Preferably, the preferred cesium phosphate salt is Cs2HPO4.

Preferably, said potassium phosphate salt(s) or cesium phosphate salt(s) is (are) chosen from KH2PO4, CsH2PO4, optionally in their hydrate form.

Preferably, said powders of at least one potassium phosphate salt and/or cesium phosphate salt can advantageously be milled and screened to a grain size of less than 100 μm prior to being introduced into step a). The grain size of the potassium phosphate salts and/or cesium phosphate salts is advantageously measured by dry laser particle size analysis.

Very preferably, said premixing step a) is advantageously carried out in a planetary centrifugal mixer. Said powders of at least one potassium phosphate salt and/or cesium phosphate salt, preferably milled and screened to a particle size of less than 100 μm, are dispersed beforehand by means of a planetary centrifugal mixer in the presence of the colloidal silica sol source so as to obtain said suspension.

Said premixing step a) is advantageously carried out at a mixing speed applied to the planetary centrifugal mixer of between 100 and 2000 revolutions per minute, preferably between 200 and 500 revolutions per minute.

Preferably, said step a) is carried out for a period of time of between 5 and 60 seconds and preferably between 20 and 60 seconds.

The additions of powders, of colloidal silica sol and of solvent can also be advantageously alternated.

### Step b):

In accordance with the invention, the process comprises a step b) of adding a powder of at least one precipitated silica and at least one solvent to said suspension obtained on conclusion of step a).

Preferably, the precipitated silicas or silica gels added in step b) are chosen, without being restricted, from the following commercial sources: Nyasil20 (Nyacol®), Siliaflash P60 (Silicycle R), Siliaflash C60 (Silicycle R), Ultrasil VN3 GR (Evonik®), taken alone or as a mixture.

Preferably, the powder of at least one precipitated silica or silica gel added in step b) has a grain size of less than 10 μm, and preferably less than 5 μm, more preferably less than 1 μm. The precipitated silica grain size is advantageously measured by dry laser particle size analysis.

Preferably, the precipitated silica is in amorphous form.

In the context of the invention, it is entirely possible to envisage mixing together several different silica powders and/or different silica sols and/or different powders of potassium phosphate salt and/or cesium phosphate salt.

According to the invention, at least one solvent is added in step b). Said solvent is advantageously chosen from water, ethanol, alcohols and amines. Preferably, said solvent is water.

Preferably, at least one organic adjuvant may also be added during step b).

Said organic adjuvant can also be chosen from all the additives known to those skilled in the art.

In the case where at least one organic adjuvant is added in step b), said organic adjuvant is advantageously chosen from cellulose derivatives, polyethylene glycols, aliphatic monocarboxylic acids, aromatic alkylated compounds, sulfonic acid salts, fatty acids, polyvinylpyrrolidone, polyvinyl alcohol, methylcellulose, polyacrylates, polymethacrylates, polyisobutene, polytetrahydrofuran, starch, polymers of polysaccharide type (such as xanthan gum), scleroglucan, derivatives of hydroxyethylcellulose type, carboxymethylcellulose, lignosulfonates and galactomannan derivatives, taken alone or as a mixture.

Preferably, said organic adjuvant can be mixed in powder form or in solution in said solvent.

The premixing step a) and the step b) can advantageously be carried out in the same piece of equipment and preferably in a planetary centrifugal mixer.

In another embodiment, the premixing step a) and the step b) can advantageously be carried out in different pieces of equipment. In this case, the premixing step a) is preferably carried out in a planetary centrifugal mixer and then the suspension obtained on conclusion of step a) is subsequently transferred into a Z-arm batch kneader in which a powder of at least one precipitated silica and at least one solvent are added to said suspension, according to step b).

In the case where step a) and step b) are not carried out in the same piece of equipment, the source of a precipitated silica, at least one solvent and optionally at least one organic adjuvant are preferably added first of all, preferably in the Z-arm batch kneader, before the suspension obtained in step a) is introduced.

In the case where a cesium phosphate salt powder is used, an addition of aqueous ammonia can be carried out so as to obtain an extrudable mixture in step b).

Preferably, said mixing step b) is carried out by batchwise or continuous kneading.

In the case where said step b) is carried out batchwise, said step b) is advantageously carried out in a kneader preferably equipped with Z-shaped arms, or a cam kneader, or in any other type of mixer, for instance a planetary mixer. Said mixing step b) makes it possible to obtain a paste or a homogeneous mixture of the constituents.

When a kneader of “Z-arm” type is used in step b), the rotation speed of the arms of the kneader is advantageously between 10 and 75 revolutions/minute, preferably between 25 and 50 revolutions/minute.

When step b) is carried out in a planetary centrifugal mixer, the rotation speed is advantageously between 300 and 2000 revolutions/minute, preferably between 1500 and 2000 revolutions/minute, so as to obtain a paste.

In one particularly preferred embodiment, the use of a powder of at least one potassium phosphate salt and/or cesium phosphate salt milled and screened to a particle size of less than 100 μm in the pre-mixing step a), combined with the use of a source of precipitated silica or silica gel having a reduced size of preferably less than 10 μm, preferably less than 5 μm, more preferably less than 1 μm, in step b), allows a significant improvement in the mechanical strength of the materials obtained according to the invention.

Preferably, the following amounts are introduced in the mixing step a) of the process according to the invention:
1% to 99% by weight, preferably from 5% to 99% by weight, preferably from 5% to 95% by weight, and very preferably from 5% to 40% by weight and even more preferably from 5% to 20% by weight of at least one colloidal silica sol,
1% to 99% by weight, preferably from 5% to 99% by weight, preferably from 10% to 95% by weight, and very preferably from 10% to 60% by weight of at least one potassium phosphate salt and/or cesium phosphate salt,
1% to 99% by weight, preferably from 5% to 99% by weight, preferably from 10% to 95% by weight, and very preferably from 30% to 80% by weight of at least one precipitated silica,
0% to 20% by weight, preferably from 1% to 15% by weight, preferably from 1% to 10% by weight, and very preferably from 1% to 7% by weight of at least one organic adjuvant, the weight percentages being expressed relative to the total weight of said material, and the sum of the contents of each of the compounds of said material being equal to 100%.

### Step c):

In accordance with the invention, said process comprises a step c) of forming into shape the paste obtained on conclusion of the mixing step b).

Preferably, the paste obtained on conclusion of step b) is advantageously formed into shape by extrusion.

When the forming into shape of the mixture resulting from step b) is carried out by extrusion, said step b) is advantageously carried out in a single-screw or twin-screw piston extruder.

In this case, an organic adjuvant can optionally be added in the mixing step b). The presence of said organic adjuvant facilitates the forming into shape by extrusion. Said organic adjuvant is described above and is introduced in step b) in the proportions indicated above.

The extrusion of the mixture, also called “kneaded paste”, can be carried out either by directly extruding at the end of a twin-screw continuous kneader for example, or by connecting one or more batch kneaders to an extruder. The geometry of the die, which gives the extrudates their shape, can be chosen from dies well known to those skilled in the art. They may thus for example be of cylindrical, multilobe, fluted or slotted shape.

In the case where the forming into shape of the mixture resulting from step b) is carried out by extrusion, the amount of solvent added in the mixing step b) is adjusted so as to obtain, on conclusion of this step and regardless of the variant implemented, a mixture or a paste which does not run but which is also not too dry, in order to allow its extrusion under suitable pressure conditions well known to those skilled in the art and dependent on the extrusion equipment used.

Preferably, said step c) of forming into shape by extrusion is carried out at an extrusion pressure greater than 1 MPa and preferably between 3 MPa and 10 MPa.

### Step d):

The preparation process according to the invention comprises a step d) of maturing the material formed into shape, obtained on conclusion of step c). Said maturation step is advantageously carried out at a temperature of between 0 and 300° C., preferably between 2° and 200° C. and very preferably between 2° and 150° C., for a time of between 1 minute and 72 hours, preferably between 30 minutes and 72 h, and preferably between 1 h and 48 h and more preferably between 1 and 24 h.

Preferably, said maturation step is carried out under air and preferably under moist air with a relative humidity of between 20 and 100% and preferably between 70 and 100%. This step allows good hydration of the material required to limit the appearance of cracks which are harmful to the mechanical strength.

Advantageously, the material formed into shape and resulting from the maturation step d) can also optionally undergo a calcining step d′) at a temperature of between 5° and 800° C., preferably between 100 and 550° C. for a period of time of between 1 and 12 h and preferably between 1 and 4 h. This calcining step is notably useful in order to eliminate the organic adjuvants used in order to facilitate the forming into shape of the material.

Said optional calcining step d′) is advantageously carried out under a gas stream comprising oxygen: for example preferably the extrudates are calcined under dry air or with various degrees of humidity or else temperature treated in the presence of a gas mixture comprising an inert gas, preferably nitrogen, and oxygen. The gas mixture used preferably comprises at least 5% by volume, or even preferably at least 10% by volume of oxygen.

### Optional Step e):

In one preferred embodiment, the process comprises a step e) of hydrothermal treatment in the presence of water vapour.

The hydrothermal treatment is performed via any technique known to those skilled in the art. The term “hydrothermal treatment” means the placing in contact, at any step in the production, of the mixed support with water in the vapour phase or in the liquid phase. The term “hydrothermal treatment” may mean notably maturing, steaming, autoclaving, calcining under moist air, rehydration. Without it reducing the scope of the invention, such a treatment has the effect of making the silica component mobile.

In one very preferred embodiment, the hydrothermal treatment step e) is carried out at atmospheric pressure, at a temperature of between 200 and 1100° C. and preferably of between 400° C. and 1000° C., for a period of time of between 30 minutes and 5 hours. The composition by volume of the water in the gas is between 5% and 100%, preferably 10% and 90%.

In another very preferred embodiment, the hydrothermal treatment step e) is carried out under a water partial pressure. The support can thus be advantageously subjected to a hydrothermal treatment in a confined atmosphere or by autoclaving. The term “hydrothermal treatment in a confined atmosphere” means a treatment by passing through an autoclave in the presence of water at a temperature above ambient temperature.

According to this very preferred embodiment of the invention, the hydrothermal treatment is a temperature treatment under a stream containing gaseous water and a gas, and under pressure. The gas is advantageously air or nitrogen. The composition by volume of the water in the gas is between 5% and 100%, preferably 10% and 90%. The temperature during the hydrothermal treatment can be between 10° and 1100° C. and preferably between 10° and 450° C. and preferably between 200° C. and 450° C., for a period of time of between 30 minutes and 24 hours and preferably between 30 minutes and 4 hours. The water partial pressure is between 0.1 and 10 MPa, preferably between 0.11 and 7.5 MPa and more preferably between 0.1 and 5 MPa.

In one preferred embodiment, said hydrothermal treatment step e) can, where appropriate, totally or partly replace the calcining step d′).

On conclusion of the process for preparing the material according to the invention, the material obtained is in the form of extrudates or of pellets.

However, it is not out of the question for said materials obtained to subsequently be, for example, introduced into a piece of equipment which allows their surface to be rounded off, such as a pan or any other piece of equipment allowing spheronization of said materials.

Said materials obtained according to the process of the invention have BET specific surface areas of between 0 and 180 m²/g.

Said materials obtained according to the process of the invention have a pore volume of between 0 and 0.55 cm3/g.

Said materials obtained according to the process of the invention have a macropore volume of between 0 and 0.31 cm3/g.

Said materials obtained according to the process of the invention have a mesopore volume of between 0 and 0.4 cm3/g.

Said preparation process according to the invention makes it possible to obtain materials according to the invention having mechanical strength values measured by single pellet crush of greater than 0.5 daN/mm, preferably greater than 0.7 daN/mm, preferably greater than 0.9 daN/mm and preferably greater than 1.3 daN/mm.

The material obtained on conclusion of the preparation process according to the invention can be used for applications in catalysis.

Said material is brought into contact with the gaseous feedstock to be treated in a reactor, which may be either a fixed-bed reactor, or a radial reactor, or else a fluidized-bed reactor.

Another subject of the present invention relates to the material that can be obtained via said process according to the invention.

### Catalytic Preparation of Acrylic Acid

One aspect included is a process for the preparation of acrylic acid comprising the steps of: i) preparing a lactic acid containing feedstock, ii) heating the feedstock to obtain a lactic acid containing vapor phase, iii) contacting the lactic acid containing vapor phase with a catalyst to create a reacted vapor phase, and iv) isolating acrylic acid from the reacted vapor phase where in the catalyst is prepared by methods disclosed herein.

A “lactic acid containing feedstock” as used herein means a solution or mixture of components containing lactic acid. The lactic acid may be included in any concentration. In some aspects the lactic acid containing feedstock is an aqueous solution. In some aspects lactic acid containing feedstock is an aqueous solution containing between 5% and 25% by weight lactic acid. In some aspects lactic acid containing feedstock is an aqueous solution containing between 15% and 85% by weight lactic acid. In some aspects lactic acid containing feedstock is an aqueous solution containing between 50% and 75% by weight lactic acid.

The lactic acid containing feedstock may then be heated in reactor to vaporization. The lactic acid containing feedstock may be dripped or injected into the preheated reactor where it will quickly vaporize. Lactic acid vaporization is known in the art and any suitable reactor or series of reactors may be utilized in the present invention. The reaction may be carried out at a temperature of between 350° C. and 400° C. and at an elevated pressure. The reaction may include the use of a carrier gas such as nitrogen or steam. The carrier gas may be added externally or prepared by the vaporization of the feedstock, or both.

The vaporized feedstock is then contacted with the catalysts of the present disclosure. These catalysts are shown to efficiently facilitate the dehydration of lactic acid to acrylic acid.

After conversion, the reaction gases are cooled, acrylic acid is condensed and isolated as the product of the reaction. The condensation and isolation of the acrylic acid product is also a well-known procedure in the art.

### EXAMPLES

The examples below illustrate the invention without limiting the scope thereof:

TABLE 1

| | | | | | % Cs ou | % | % | % KH2PO4 |
|---|---|---|---|---|---|---|---|---|
| | | Source of | | | K/KPO3 | Powdered | colloïdal | or |
| | Catalyst | K ou Cs | MEF | Broyage | visé | silica | silica sol | CsH2PO4 |
| example 1 | A1 | KH₂PO₄ | MP + BR | yes | 22.18% | 64.31 | 10.97 | 24.73 |
| example 2 | A2 | KH₂PO₄ | all MP | yes | 22.26% | 64.22 | 10.97 | 24.81 |
| example 3 | A3 | KH₂PO₄ | all MP | yes | 28.46% | 60.05 | 8.52 | 31.44 |
| example 4 | A4 | KH₂PO₄ | all MP | yes | 46.46% | 35 | 15 | 50 |
| example 5 | A5 | CsH₂PO₄ | MP + BR | yes | 13.05% | 74 | 12 | 14 |
| example 6 | A6 | CsH₂PO₄ | MP + BR | yes | 33.17% | 56 | 9 | 35 |
| example 7 | A7 | CsH₂PO₄ | all MP | yes | 13.05% | 74 | 12 | 14 |

| | | | V | D at V | V | D a V | Extrude |
|---|---|---|---|---|---|---|---|
| | S_{BET} | TPV_{Hg} | macro | macro/2 | meso | meso/2 | mean SPC |
| | (m²/g) | (ml/g) | (ml/g) | (nm) | (ml/g) | (nm) | (daN/mm) |
| example 1 | 46 | 0.28 | 0.06 | 102 | 0.19 | 13.08 | 1.02 |
| example 2 | 46 | 0.36 | 0.17 | 350 | 0.14 | 11.63 | 1.15 |
| example 3 | 26 | 0.25 | 0.08 | 324 | 0.12 | 12.35 | 1.43 |
| example 4 | 1.000 | 0.29 | 0.24 | 440 | 0 | 9.99 | 1.55 |
| example 5 | 93 | 0.27 | 0.01 | 388 | 0.24 | 9.76 | 0.6 |
| example 6 | 22 | 0.17 | 0.01 | 808 | 0.14 | 16.52 | 1.9 |
| example 7 | 92 | 0.28 | 0.01 | 432 | 0.25 | 9.94 | 0.81 |

### Example 1 According to the Invention: Premixing in a Planetary Mixer then Kneading in a Z-Arm Kneader and Forming in² to Shape by Extrusion

A source of colloidal silica sol (10.97%) and of potassium dihydrogen phosphate (KH2PO4: Aldrich) (24.72%) powder milled and screened to 100 μm are introduced into and mixed in the container of a Thinky planetary centrifugal mixer. The rotation speed is set at 1500 rpm for 30 s. The suspension obtained, a methocel (K15M) powder (3%) and a precipitated silica powder (Nyasil20; Nyacol) (64.31%), are introduced into and premixed in a Brabender kneader. The water is added dropwise until a paste is obtained and the kneading is continued for 20 minutes. The paste obtained is then extruded on an MTS piston extruder using a cylindrical die with a diameter of 1.6 mm. The extrudates are dried for 16 h at 120° C. in a ventilated oven and then calcined for 4 h at 450° C.

The extrudates obtained have an SPC value of 1.02 daN/mm and an SBET of 46 m²/g, a TPV of 0.28 cm3/g with a macropore volume of 0.06 cm3/g and a mesopore volume of 0.19 cm3/g and a Dmacro=102 nm. The material obtained has a ratio KPO3/(KPO3+SiO₂)=22.2%.

### Example 2 According to the Invention: Step a) and Step b) in a Planetary Centrifugal Mixer then Forming into Shape by Extrusion

A source of colloidal silica sol (10.97%) and of potassium dihydrogen phosphate (KH2PO4: Aldrich) (24.81%) powder milled and screened to 100 μm are introduced into and mixed in the container of a Thinky planetary centrifugal mixer. The rotation speed is set at 1500 rpm for 30 s. A methocel (K15M) powder (3%) and a precipitated silica powder (Nyasil20: Nyacol) (64.22%) are added, in the container of the planetary centrifugal mixer, to the suspension obtained. The rotation speed is set at 2000 rpm for 30 s. The paste obtained is then extruded on an MTS piston extruder using a cylindrical die with a diameter of 1.6 mm. The extrudates are dried for 16 h at 120° C. in a ventilated oven and then calcined for 4 h at 450° C.

The extrudates obtained have an SPC value of 1.15 daN/mm and an SBET of 46 m²/g, a TPV of 0.36 cm3/g with a macropore volume of 0.17 cm3/g and a mesopore volume of 0.14 cm3/g and a Dmacro=350 nm. The material obtained has a ratio KPO3/(KPO3+SiO2)=22.2%.

### Example 3 According to the Invention: Step a) and Step b) in a Planetary Centrifugal Mixer with a High Potassium Phosphate Content then Forming into Shape by Extrusion

A source of colloidal sol (8.52%) and of potassium dihydrogen phosphate (KH2PO4: Aldrich) (31.44%) powder milled and screened to 100 μm are introduced into and mixed in the container of a Thinky planetary centrifugal mixer. The rotation speed is set at 1500 rpm for 30 s. A methocel (K15M) powder (3%) and a precipitated silica powder (Nyasil20): Nyacol) (60.05%) are added, in the container of the planetary centrifugal mixer, to the suspension obtained. The rotation speed is set at 2000 rpm for 30 s. The paste obtained is then extruded on an MTS piston extruder using a cylindrical die with a diameter of 1.6 mm.

The extrudates are dried for 16 h at 120° C. in a ventilated oven and then calcined for 4 h at 450° C.

The extrudates obtained have an SPC value of 1.43 daN/mm and an SBET of 26 m²/g, a TPV of 0.25 cm3/g with a macropore volume of 0.08 cm3/g and a mesopore volume of 0.12 cm3/g and a Dmacro=324 nm. The material obtained has a ratio KPO3/(KPO3+SiO2)=28.5%.

### Example 4 According to the Invention: Step a) and Step b) in a Planetary Centrifugal Mixer with a High Potassium Phosphate Content then Forming into Shape by Extrusion

A source of colloidal sol (15%) and of potassium dihydrogen phosphate (KH2PO4: Aldrich) (50%) powder milled and screened to 100 μm are introduced into and mixed in the container of a Thinky planetary centrifugal mixer. The rotation speed is set at 1500 rpm for 30 s. A methocel (K15M) powder (3%) and a precipitated silica powder (Nyasil20); Nyacol) (35%) are added, in the container of the planetary centrifugal mixer, to the suspension obtained. The rotation speed is set at 2000 rpm for 30 s. The paste obtained is then extruded on an MTS piston extruder using a cylindrical die with a diameter of 1.6 mm.

The extrudates are dried for 16 h at 120° C. in a ventilated oven and then calcined for 4 h at 450° C.

The extrudates obtained have an SPC value of 1.55 daN/mm and an SBET<1 m²/g, a TPV of 0.29 cm3/g with a macropore volume of 0.24 cm3/g and a mesopore volume of 0 cm3/g and a Dmacro=440 nm. The material obtained has a ratio KPO3/(KPO3+SiO2)=46.5%.

### Example 5 According to the Invention: Premixing Step a) in a Planetary Mixer Using a Cesium Phosphate Salt then Step b) in a Z-Arm Kneader and Forming into Shape by Extrusion

A source of colloidal silica sol (12%) and of cesium dihydrogen phosphate (CsH2PO4: Alfa Chemistry) (14%) powder milled and screened to 100 μm are introduced into and mixed in the container of a Thinky planetary centrifugal mixer. The rotation speed is set at 1500 rpm for 30 s. A methocel (K15M) powder (3%) and a precipitated silica powder (Nyasil20; Nyacol) (74%) are introduced into the suspension obtained and premixed in a Brabender kneader. The water is added dropwise until a paste is obtained and the kneading is continued for 20 minutes. The paste obtained is then extruded on an MTS piston extruder using a cylindrical die with a diameter of 1.6 mm.

The extrudates are dried for 16 h at 120° C. in a ventilated oven and then calcined for 4 h at 450° C.

The extrudates obtained have an SPC value of 0.6 daN/mm and an SBET of 93 m²/g, a TPV of 0.27 cm3/g with a macropore volume of 0.01 cm3/g and a mesopore volume of 0.24 cm3/g and a Dmacro=388 nm. The material obtained has a ratio CsPO3/(CsPO3+SiO2)=13%.

### Example 6 According to the Invention: Premixing Step a) in a Planetary Mixer Using a High Cesium Phosphate Salt Content then Step b) in a Z-Arm Kneader and Forming into Shape by Extrusion

A source of colloidal silica sol (9%) and of cesium dihydrogen phosphate (CsH2PO4; Alfa Chemistry) (35%) powder milled and screened to 100 μm are introduced into and mixed in the container of a Thinky planetary centrifugal mixer. The rotation speed is set at 1500 rpm for 30 s. A methocel (K15M) powder (3%) and a precipitated silica powder (Nyasil20): Nyacol) (56%) are introduced into the suspension obtained and premixed in a Brabender kneader. The water to which aqueous ammonia has been added is added dropwise until a paste is obtained and the kneading is continued for 20 minutes. The paste obtained is then extruded on an MTS piston extruder using a cylindrical die with a diameter of 1.6 mm.

The extrudates are dried for 16 h at 120° C. in a ventilated oven and then calcined for 4 h at 450° C.

The extrudates obtained have an SPC value of 1.9 daN/mm and an SBET of 22 m²/g, a TPV of 0.17 cm3/g with a macropore volume of 0.01 cm3/g and a mesopore volume of 0.14 cm3/g and a Dmacro=808 nm. The material obtained has a ratio CsPO3/(CsPO3+SiO2)=33.2%.

### Example 7 According to the Invention: Step a) and Step b) in a Planetary Centrifugal Mixer Using a Cesium Phosphate Salt then Forming into Shape by Extrusion

A source of colloidal silica sol (12%) and of cesium dihydrogen phosphate (CsH2PO4; Alfa Chemistry) (14%) powder milled and screened to 100 μm are introduced into and mixed in the container of a Thinky planetary centrifugal mixer. The rotation speed is set at 1500 rpm for 30 s. A methocel (K15M) powder (3%) and a precipitated silica powder (Nyasil20; Nyacol) (74%) are added, in the container of the planetary centrifugal mixer, to the suspension obtained. The rotation speed is set at 2000 rpm for 30 s. The paste obtained is then extruded on an MTS piston extruder using a cylindrical die with a diameter of 1.6 mm. The extrudates are dried for 16 h at 120° C. in a ventilated oven and then calcined for 4 h at 450° C.

The extrudates obtained have an SPC value of 0.81 daN/mm and an SBET of 92 m²/g, a TPV of 0.28 cm3/g with a macropore volume of 0.01 cm3/g and a mesopore volume of 0.25 cm3/g and a Dmacro=432 nm. The material obtained has a ratio CsPO3/(CsPO3+SiO2)=13%.

### COMPARATIVE EXAMPLES

The powdered catalysts A8 and A9 (Comparative examples) were prepared according to patent WO17040383 (U.S. Pat. No. 9,849,447). The materials are prepared by mechanical mixing, by means of a planetary mill, of an amorphous fused silica which is a dense material with neither surface nor porosity properties, and of potassium phosphate precursors. The catalyst A8 calcined under air at 450° C. is composed of a KPO3/(KPO3+SiO2) mixture with a mass ratio of 26 wt % of KPO3 according to example 8 of the patent. The catalyst A9 calcined under air at 450° C. is composed of a CsPO3/(CsPO3+SiO2) mixture with a mass ratio of 39 wt % of CsPO3 according to procedure described in example 12 of the patent, but with higher amount of CsPO3.

The catalysts were tested in two experimental devices.

### SET UP A-16 Reactors Set Up Brief Description

The catalysts are tested in an Avantium device with 16 fix beds reactors in parallel. The inner quartz reactor diameter is 2 mm with a length of 560 mm. The liquid and the gas are distributed at the inlet of the reactor and mixed before introduction. The evaporation of the feed is carried out at the head of the reactor with an absorbent material (wick).

After reactor, all the products are analyzed online by gas chromatography.

### SET UP B-4 Reactors Set Up Brief Description

The catalysts were tested in an Avantium apparatus with 4 fixed bed reactors in parallel. The internal quartz reactor diameter is 2 mm or 4 mm with a length of 560 mm. The liquid and the gas are distributed at the inlet of the reactor and mixed before introduction. The evaporation of the feed is carried out at the head of the reactor with an absorbent material (wick).

After reactor, the gas phase is cooled and condensed. The liquid is collected at atmospheric pressure and at 10° C. and further analyzed by HPLC. The gas phase is analyzed online by gas chromatography.

### Example A1: Catalytic Performances in a 16 Reactors Device (SET UP A)

The catalysts are tested in the device of 16 reactors in parallel (SET UP A).

The volume of catalyst loaded is 0.2 mL. The powdered and the shaped catalysts are loaded into the tube with an internal diameter of 2 mm. The reaction is carried out in gas phase, at a temperature of 375° C. and a pressure of 10 barr. The feed is a 12 wt. % lactic acid in water. The weight hourly space velocity of lactic acid is in between 0.2 and 0.25 h-1 with a mass of catalyst loading between 120 to 200 mg.

AA yield is determined by GC analysis at 20 h run time.

TABLE 2

| | | MPO3/ | | Bed | m | AA |
|---|---|---|---|---|---|---|
| Catalyst | Example | (MPO3 + SiO2) | | high | catalyst | yield % |
| Label | patent | (% pds.) | Shape | cm | g | molC |
| A1 | Ex 1 | 22.18% KPO3 | Extudates 1.6 | 55 | 144 | 81 |
| A2 | Ex 2 | 22.26% KPO3 | Extudates 1.6 mm | 55 | 170 | 79 |
| A3 | Ex 3 | 28.46% KPO3 | Extudates 1.6 mm | 55 | 173 | 81 |
| A4 | Ex 4 | 46.5% KPO3 | Extudates 1.6 mm | 55 | 165 | 82 |
| A5 | Ex 5 | 13.05% CsPO3 | Extudates 1.6 | 55 | 181 | 59 |
| A6 | Ex 6 | 33.15% CsPO3 | Extudates 1.6 mm | 55 | 217 | 78 |
| A7 | Ex 7 | 13.05% CsPO3 | Extudates 1.6 mm | 55 | 191 | 71 |
| A8 | — | 26% KPO3 | Powder 100-250 | 55 | 152 | 81 |
| Comparative | | | microns | | | |
| example | | | | | | |
| A9 | — | 37% CsPO3 | Powder 100-250 | 55 | 199 | 70 |
| Comparative | | | microns | | | |
| example | | | | | | |

Catalysts A1 to A9 are active and selective in converting lactic acid to acrylic acid, with carbon yield higher than 70%. The unloading of shaped catalysts A1 to A7 is easier than for powdered catalysts A8 and A9. Indeed, the powder is stuck to the wall of the quartz reactor. An advantage of using a shaped catalyst is the easier way to unload it.

### Example 2: Catalytic Performance in a Device Comprising 4 Reactors in Parallel (SET UP B)

The catalysts are tested in the device comprising 4 fixed bed reactors in parallel with different operating conditions from example 1: higher volume of catalyst, higher total pressure, and different analytical setup.

The volume of catalyst loaded is 1 mL. The powdered catalyst is loaded into the 4.00 mm ID tubes while the shaped catalysts are loaded into the 2 mm ID tubes. The reaction is carried out in gas phase, temperature of 375° C., a pressure of 25 barg. The feed is a 20 wt. % lactic acid in water. The liquid hourly space velocity is 1.2 h-1. The nitrogen flowrate is 25 Nml/min. The gas hourly space velocity is 3000 h-1.

The conversion of lactic acid is defined as follows:

$$LA conversion = 100 \times ( LA in - LA out ( \% pds ) ) / LA in$$

With LA: LA concentration in liquid phase from feed and sample (HPLC analysis) g/l

$$AA yield ( \% mol C ) = 100 \times ( AA out ) / ( LA in - LA out )$$

LA in and LA out: LA concentration in the liquid phase from feed and sample (HPLC analysis) mol C/l
AA: AA concentration in liquid phase of the sample (HPLC analysis) mol C/l

The catalytic performances are given for a TOS of 18 h.

TABLE 3

| | MPO3/ | | | m | | AA |
|---|---|---|---|---|---|---|
| | (MPO3 + SiO2) | | V | catalyst | LA | yield % |
| | (% pds.) | Shape | catalyst | g | conversion % | molC |
| A1 | 22.18% KPO3 | Extrudates | 0.9 mL | 0.580 | 99.0 | 64 |
| | | 1.6 mm | | | | |
| A8 | 26% KPO3 | Powder | 0.9 mL | 0.690 | 99.4 | 63 |
| | | 100-250 | | | | |
| | | microns | | | | |
| A6 | 33.15% CsPO3 | Extrudates | 0.9 mL | 0.720 | 98.2 | 63 |
| | | 1.6 mm | | | | |
| A9 | 39% CsPO3 | Powder | 0.9 mL | 0.912 | 99.6 | 36 |
| | | 100-250 | | | | |
| | | microns | | | | |

Catalysts A1, A6, A8 and A9 are active and selective in converting lactic acid to acrylic acid, with a carbon yield higher than 60%. The conversion of lactic acid is always above 99%.

Unloading shaped catalyst A1 and A6 is less difficult than discharging powdered catalysts A7 and A8. Powdered catalyst have the potential to adhere to the walls of the reactor. Therefore, the shaped catalyst has a distinct advantage when it comes to unloading.

1. Process for the preparation of acrylic acid comprising the steps of: i) preparing a lactic acid containing feedstock, ii) heating the feedstock to obtain a lactic acid containing vapor phase, iii) contacting the lactic acid containing vapor phase with a catalyst to create a reacted vapor phase, and iv) isolating acrylic acid from the reacted vapor phase where in the catalyst is prepared by the following process:
a) a step of premixing at least one colloidal silica sol with at least one powder of at least one potassium phosphate salt and/or cesium phosphate salt, so as to obtain a suspension,
b) a step of adding a powder of at least one precipitated silica and at least one solvent to said suspension obtained on conclusion of step a),
c) a step of forming into shape the mixture obtained on conclusion of step b),
d) a step of maturing the material obtained on conclusion of step c).

2. Process according to claim 1; in which the potassium phosphate salts are chosen from the following list: KH₂PO₄, KH₂P₂O₁₂, K₆P₆O₇, K₃H₂P₃O₁₀, K₄H₂P₄O₁₃, K₃P₃O₉, K₄P₄O₁₂, K₆P₆O₁₈, K₈P₈O₂₄, K₁₀P₁₀O₃₀, potassium (tripotassium) phosphate (PO₄³⁻, 3K⁺), alone or as a mixture, and preferably the potassium phosphate salt is KH₂PO₄.

3. Process according to claim 2 in which the cesium phosphate salts are chosen from the following list: CsH₂PO₄, Cs₂H₂P₃O₁₀, Cs₄H₂P₄O₁₃, Cs₃P₃O₉, Cs₄P₄O₁₂, Cs₆P₆O₁₈, Cs₈P₈O₂₄, (CsPO₃), alone or as a mixture, preferably the cesium phosphate salt is Cs₂HPO₄.

4. Process according to claim 2 in which, prior to their introduction in step a), said powders of at least one potassium phosphate salt and/or cesium phosphate salt are milled and screened to a grain size of less than 100 μm.

5. Process according to claim 3 in which said pre-mixing step a) is carried out in a planetary centrifugal mixer at a mixing speed applied to the planetary centrifugal mixer of between 100 and 2000 revolutions per minute.

6. Process according to claim 4 in which said solvent added in step b) is chosen from water, ethanol, alcohols and amines, and preferably said solvent is water.

7. (canceled)

8. (canceled)

9. (canceled)

10. (canceled)

11. Process according claim 6 in which said maturation step is carried out at a temperature of between 20° C. and 150° C., for a time of between 1 hour and 48 hours.

12. Process according to claim 11 in which the material formed into shape and resulting from the maturation step c) undergoes a calcining step d) at a temperature of between 100° C. and 550° C. for a period of time of between 1 hour and 4 hours.

13. Process according to claim 12 in which said process comprises a step e) of hydrothermal treatment in the presence of water vapour, it being possible for step e) to be carried out under atmospheric pressure or under a water partial pressure.

14. Process according to claim 13 in which the hydrothermal treatment step e) is carried out at atmospheric pressure, at a temperature of between 400° C. and 1000° C., for a period of time of between 30 minutes and 5 hours, the composition by volume of water in the gas in said step d) being between 10% and 90%.

15. Process according to claim 14; in which the hydrothermal treatment step e) is carried out at a temperature of between 200° C. and 450° C., for a period of time of between 30 minutes and 4 hours and under a water partial pressure of between 0.11 and 7.5 MPa.

16. Process according to claim 1, wherein process is performed at a temperature of between 350° C. and 400° C. and the feedstock contains 15% to 85% lactic acid by weight.
